(19) United States
(12) Patent Application Publication
(10) Pub. No.: US 2015/0148710 A1
(43) Pub. Date: May 28, 2015

(54) Ultrasound Modulation of the Brain for Treatment of Stroke, Brain Injury, and Other Neurological Disorders

(71) Applicant: ARIZONA BOARD OF REGENTS FOR AND ON BEHALF OF ARIZONA STATE UNIVERSITY, Scottsdale, AZ (US)

(72) Inventors: Bruce C. Towe, Mesa, AZ (US); Jeffrey Kleim, Scottsdale, AZ (US)

(73) Assignee: ARIZONA BOARD OF REGENTS ON BEHALF OF ARIZONA STATE UNIVERSITY, Scottsdale, AZ (US)

(21) Appl. No.: 14/399,813

(22) PCT Filed: Mar. 14, 2013
(86) PCT No.: PCT/US2013/031355

§ 371 Date: Nov. 7, 2014

Related U.S. Application Data
(60) Provisional application No. 61/643,747, filed on May 7, 2012.

(51) Int. Cl. A61N 7/00

(52) U.S. Cl. CPC A61N 7/00; A61N 2007/0026
USPC 601/2

(57) ABSTRACT

A method for ultrasound modulation of the brain for treatment of stroke, brain injury, and other neurological disorders or the improvement of cognitive functioning in patients. The method may include identifying a stimulation site of a brain, where the stimulation site is associated with a brain disorder, applying ultrasound to the stimulation site, and initiating physical therapy. Alternately, ultrasound may be applied to the brain to enhance aspects of cognitive functioning by the combination of exercising the functionality of the brain and applying ultrasound to the region(s) or structures of the brain known to be associated with that function.

### CROSS-REFERENCE TO RELATED APPLICATIONS

This application claims priority to U.S. Provisional Patent Application No. 61/643,747, filed May 7, 2012, hereby incorporated by reference in its entirety.

### BACKGROUND OF THE INVENTION

1. Field of the Invention

This invention relates to ultrasound modulation of the brain and more particularly relates to a method for ultrasound modulation of the brain for treatment of stroke, brain injury, and other neurological disorders.

2. Description of the Related Art

Causes of brain damage include stroke, traumatic brain injury (TBI), and other disorders of the brain that require the brain to remap its function and organization. Physical therapy is generally the standard treatment for brain injury. However it is a long, tedious, and expensive process whose outcome is often far from satisfactory.

Electrical stimulation applied to the brain cortex for treatment of brain damage has been under investigation for many years and it has been shown to be effective in improving patient outcomes of physical therapy. The exact mechanism of its therapeutic effect in the brain is unknown but thought to be a result of producing a subthreshold excitation of brain cells that enables them to more readily change in their synaptic connections and functionality. The effects may include facilitated learning and may last for long periods of time.

Electrical energy however needs to be applied locally to the brain tissue to areas that are relearning with therapy. Reported electrical methods such as those of Northstar Inc. generally require electrodes to be directly placed locally on or near portions of the brain that are expected to change in their function by remapping.

The process of brain remapping for lost function is evoked by patient physical therapy. Other approaches include transcranial magnetic stimulation (TMS) that, although noninvasive, fails to provide the needed localization of induced electrical stimulatory effects on the brain. Studies have shown that TMS is able to produce a lasting change in neural activity within the cortex that persists after terminating the treatment. Transcranial electrical stimulation (TES) also cannot provide location-specific current delivery in the brain and may also produce pain.

Functional changes that occur in the brain are a result of a re-learning nature of brain cells to perform new functions, called neural plasticity. There is an invasiveness to electrical devices that limits their application because highly invasive surgery is required to access the brain.

Biological tissues are known to be responsive to other forms of applied energy. It is known that ultrasound energy has effects on the brain and nervous tissues. For example, U.S. Pat. No. 7,702,395 to Towe et al. discloses an application of high frequency ultrasound energy used in medical imaging that affect changes in neural excitability. Ultrasound at considerably lower frequencies than used in ultrasound medical imaging has bioelectrical effects on the brain as evidenced by the production of muscle action events.

Ultrasound applications have been described in the patent literature for bioelectrical stimulatory effects on the brain. The reason for the ultrasound effectiveness in creating neural effects is a matter of current scientific debate. A popular theory is that there is a type of cell membrane stretch activation that results from the compression-rarefaction nature of ultrasound; another is that there is a cavitation or stirring effect that affects sodium channels.

There are also different effects on excitable tissues depending on the ultrasound pulse protocol. For example on nerve axons the effects of relatively slowly repeating ultrasound pulses of high peak amplitude effects are inhibitory where the effects of high amplitude fast repeating ultrasound pulses are excitatory.

Stroke is neurological damage resulting from a restricted blood flow to the brain caused by emboli, hemorrhages, or clotting that causes a diminished brain function according to the function of the region where the blood flows. Surviving patients are usually subjected to physical therapy to retrain healthy parts of the brain to regain some loss of function of a limb or another affected body part. By repetitive physical motion of limbs, practicing speech, or other training methods, undamaged parts of the brain re-map at least some of their functionality to restore lost function. Unfortunately, physical therapy is a long process and is not always effective.

It is known that applying electrical energy to the brain can affect its relearning after brain damage. For example, low levels of TMS stimulation, where movement was not induced in neuro-block models that mimic amputation, is able to modify the lasting changes in neural activity that normally accompany amputation.

Electrical stimulation methods have been used for accelerating the process of stroke rehabilitation. This is performed by applying electrical currents to brain regions surrounding the stroked area and in combination with a physical therapy whereby the patient attempts to exercise the lost functionality.

Electrical current applied to brain tissue, even when applied to regions that are neuroplastic, however is not sufficient to achieve therapeutic benefits. Rather it is a combination of applying the electrical energy with physical therapy whereby the patient actively attempts to exercise lost function such as muscle control, speech, or other body function. One problem with application of electrical energy is that currents must be localized and so this requires invasive techniques. This is typically performed through the use of electrode arrays implanted in the brain connected to an implanted or external electrical pulse generator. Implantation of these devices involves trauma to the patient, risks of surgery, and high expense. The device may not be easily removable when the process of neuroplastic change is complete.

Local electrical application to the brain also requires a relatively high current drain on batteries to stimulate a relatively large amounts of cortex; depending on the tissue volume that is being treated. This lends to weight and bulk to any implantable neuroprosthesis making it uncomfortable and even impractical in some cases.

### SUMMARY OF THE INVENTION

A method is presented for ultrasound therapy of the brain for treatment of stroke, brain injury, and other neurological disorders. In some embodiments, the method includes identifying a stimulation site of a brain, where the stimulation site is associated with a brain disorder, applying ultrasound to the stimulation site, and initiating exercise of the parts of the brain involved, such as through physical therapy. In some embodiments, identifying the stimulation site of the brain may include providing a diagnostic brain image of a patient. Furthermore, identifying the stimulation site of the brain may include selecting a stimulation site from the diagnostic brain image.

In some embodiments, identifying the stimulation site of the brain may include assessing a symptom, functional change, or functional characteristic associated with the brain (e.g. that can be correlated to the known structure-function relationships of the brain) to identify the stimulation site. In some embodiments, applying ultrasound to the stimulation site may include varying ultrasound parameters. This application of ultrasound in combination with a corresponding therapy to the patient may be effective in producing neuroplastic events. The exercise of the targeted brain functionality may be invoked coincident with the application of ultrasound and continue while the ultrasound is in continuous application according to the above parameters. In some embodiments, the exercise may be applied following the ultrasound application over a duration of, for example, 1 minute to 10 minutes. The exercise of the faculty may also follow immediately after cessation of the ultrasound.

In some embodiments, applying ultrasound to the stimulation site may include applying a plurality of ultrasound generators to the patient's head proximate to the stimulation site. In addition, applying ultrasound to the stimulation site may include focusing ultrasound beams such that the ultrasound beams intersect at the stimulation site. In some embodiments, applying ultrasound to the stimulation site may include implanting ultrasound transducers under the scalp of the patient. Furthermore, applying ultrasound to the stimulation site may include implanting transducers under the patient's skull.

In some embodiments, applying ultrasound to the stimulation site may include providing ultrasound in the frequency range of 100 kHz to 1 MHz, which may transfer the sound noninvasively through the skull. In some embodiments, ultrasound may be applied at frequencies of 1 MHz to 10 MHz. In some embodiments, an ultrasound transducer may be placed external to the scalp, under the scalp, and/or under the skull bone. In addition, applying ultrasound to the stimulation site may include providing ultrasound pulses having a duration in the range of 10 milliseconds to 1000 milliseconds, and may have a duration in the range of 10 milliseconds to 300 milliseconds and have pulse duty cycles on the order of 1-10%, for example, such that the mechanical index (MI) of the ultrasound and thermal index (TI) of ultrasound may be within legal regulatory limits.

In some embodiments, applying ultrasound to the stimulation site may include repetition rates in the range of 0.1 Hz to 100 Hz, 1 Hz to 30 Hz, or 5 Hz to 10 Hz. In addition, in some embodiments, applying ultrasound to the stimulation site may include applying ultrasound in sets of pulses, where the pulse duration is between 100 milliseconds to 200 milliseconds long and the sets are repeated at a rate of 0.01 Hz to 25 Hz for durations as short as one group of pulses to continuous duty as long as the exercise of the brain continues.

In some embodiments, applying ultrasound to the stimulation site provides an ultrasound spatial peak temporal average (SPTA) amplitude greater than 0.01 watt/cm2 and less than or equal to 180 mW/cm2. In some embodiments, applying ultrasound to the stimulation site may raise the expected resting potential of a population of neurons at the stimulation site by approximately 10% to 80% of a difference between the expected resting potential and an action potential for the population of neurons.

In some embodiments, applying ultrasound to the stimulation site may include applying ultrasound stimulation to the motor cortex sufficient to cause a physically noticeable motor activity. In addition, applying ultrasound to the cortical stimulation site may include reducing the power of the ultrasound stimulation by a predetermined amount and then exercising the desired faculty in accordance with the procedure outlined above. For example, the predetermined amount may be 50% from its peak value that directly stimulates the faculty. In another embodiment, ultrasound is applied coincident with the patient strongly imagining physical movement of the affected portion of the body, which may be used if actual movement is not possible.

In some embodiments, initiating the exercise occurs later in time than applying ultrasound to the stimulation site.

The term “coupled” is defined as connected, although not necessarily directly, and not necessarily mechanically.

The terms “a” and “an” are defined as one or more unless this disclosure explicitly requires otherwise.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements possesses those one or more steps or elements, but is not limited to possessing only those one or more elements. Likewise, a step of a method or an element of a device that “comprises,” “has,” “includes” or “contains” one or more features possesses those one or more features, but is not limited to possessing only those one or more features. Furthermore, a device or structure that is configured in a certain way is configured in at least that way, but may also be configured in ways that are not listed.

Other features and associated advantages will become apparent with reference to the following detailed description of specific embodiments in connection with the accompanying drawings.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a flow chart illustrating one embodiment of a method for providing ultrasound modulation of the brain for treatment of stroke, brain injury, and other neurological disorders.

FIG. 2 is a flow chart illustrating one embodiment for identifying a stimulation site.

FIG. 3 is a flow chart illustrating another embodiment for identifying a stimulation site.

FIG. 4 is a schematic block diagram illustrating a method for providing ultrasound modulation of the brain.

FIG. 5 is a graph showing ultrasound pulse durations and repetition rates.

FIG. 6 is a graph showing the amplitude applied in some embodiments for providing ultrasound modulation of the brain.

### DETAILED DESCRIPTION

Various features and advantageous details are explained more fully with reference to the nonlimiting embodiments that are illustrated in the accompanying drawings and detailed in the following description. Descriptions of well known starting materials, processing techniques, components, and equipment are omitted so as not to unnecessarily obscure the invention in detail. It should be understood, however, that the detailed description and the specific examples, while indicating embodiments of the invention, are given by way of illustration only, and not by way of limitation. Various substitutions, modifications, additions, and/or rearrangements within the spirit and/or scope of the underlying inventive concept will become apparent to those skilled in the art from this disclosure.

Therapeutic methods are disclosed that do not apply electrical current directly to the brain. Moderate levels of ultrasound energy applied to the brain concurrent with physical therapy can be used as a method to accelerate the remapping of the brain to more rapidly recover lost neurological function. The ultrasound energy and physical therapy may promote brain plasticity.

The physiologic mechanism by which ultrasound affects this remapping process is unknown. It may be for example that ultrasound affects brain cells through pressure wave stirring and flexure movements at the fundamental level of the cell membrane where remapping and remodeling events ultimately occur. Stretch activation processes may occur at the membrane and initiate cellular processes through intermediary bioelectrical events that are dedicated to creating new connections and functionality of the cell. It is believed that ultrasound effect is intrinsically inhibitory while at higher duty cycles there is a transient thermal rise in tissue that becomes excitatory. Thus it is possible to apply ultrasound in multiple ways to excitable tissue to cause different outcomes.

Some of the methods disclosed require an ability to localize the brain region that is candidate for ultrasound-promoted neural plasticity. This may be defined by a physician but typically is at the edges of the damaged region of the brain. The indiscriminate application of ultrasound to the brain globally, or in a focused manner at a specific location will be ineffective except when the brain is exercising the functionality associated with the specific location. Physical therapy activates portions of the brain, for example, the motor and pre-motor cortex to be receptive to remodeling. These exercises may be guided by a therapist, guided by robots, the patient, or even where the patient strongly imagines to be utilizing the lost function. The combination of ultrasound therapy and physical therapy may promote improved brain remapping. In other applications, accelerated remapping of the brain may be desirable as a type of learning tool and in order to promote its enhanced functioning through ultrasound application. This may make up for deficits that are associated with pathology or promote the improved functionality of an otherwise normal brain. For example, application of ultrasound to the frontal regions of the brain may be used in combination with desirably intense voluntary exercise of this part of the brain. Such exercises will then lead to neuroplastic changes leading to increased rate of learning, improved attention, and/or improved memory as these are known functions of this part of the brain.

As a method of treating brain damage, this may require ultrasound application to the parts of the brain that will naturally remap in function to take over the function of the parts of the brain that were lost to damage. The determination of the damaged parts of the brain is generally performed by imaging such as PET, MR, CT, etc. The physician then uses his experience and taking into account the known ways that the brain recovers from such damage in order to apply ultrasound to still-functional parts of the brain where re-mapping is occurring. The ultrasound is not causing the remapping of the brain but rather promoting a natural neuroplastic process.

Some of the methods disclosed herein also monitor the effects of pulsed ultrasound on the brain as a method and tool useful in determining the ultrasound intensity and pulse parameters needed to be effective. It employs ultrasound evoked neural field potentials. Changes in brain neural field potentials are associated with a class of bioelectric responses known as evoked responses. There are for example, visual, auditory, somatosensory evoked bioelectrical responses monitored from the brain that result from corresponding stimuli to these senses.

The ultrasound energy pulse protocols needed to achieve neural plasticity effects may additionally use a dose reference point of pulse parameters required to achieve specific action events in the brain, such as evoked skeletal muscle events. After a skeletal muscle event is detected in response to an amplitude of ultrasound stimulation, the ultrasound energy may be reduced by 40% to 80%, for example, to achieve therapeutic neuroplastic effects.

The schematic flow chart diagrams that follow are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

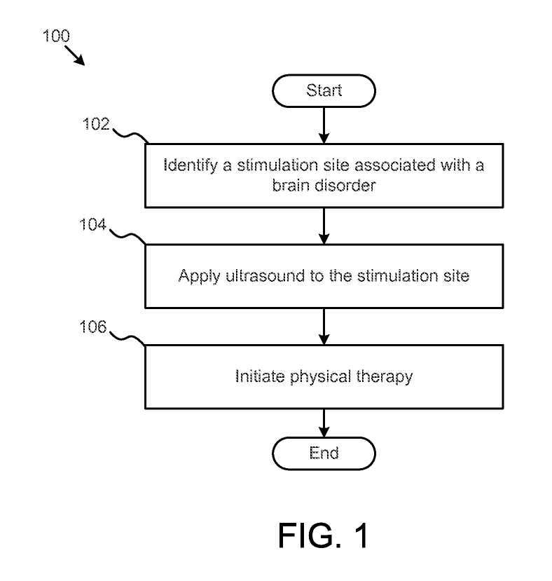

FIG. 1 shows method 100 for promoting brain remapping. The method 100 begins with 102 identification of a stimulation site associated with a brain disorder. In some embodiments the stimulation site may be the portion of the brain that has suffered pathology, but in some embodiments the stimulation site may be a portion of the brain that is learning to perform functions previously performed by the damaged portion. After the stimulation site is identified, the method 100 applies ultrasound to the stimulation site 104. Ultrasound may be provided, for example, by a piezoelectric transducer. Method 100 also includes the step of initiating physical therapy 106. In some embodiments, the physical therapy is provided simultaneously with the ultrasound, while in other embodiments the physical therapy may be provided after the ultrasound energy. For example, five minutes or more may elapse between the application of ultrasound to the stimulation site and the physical therapy.

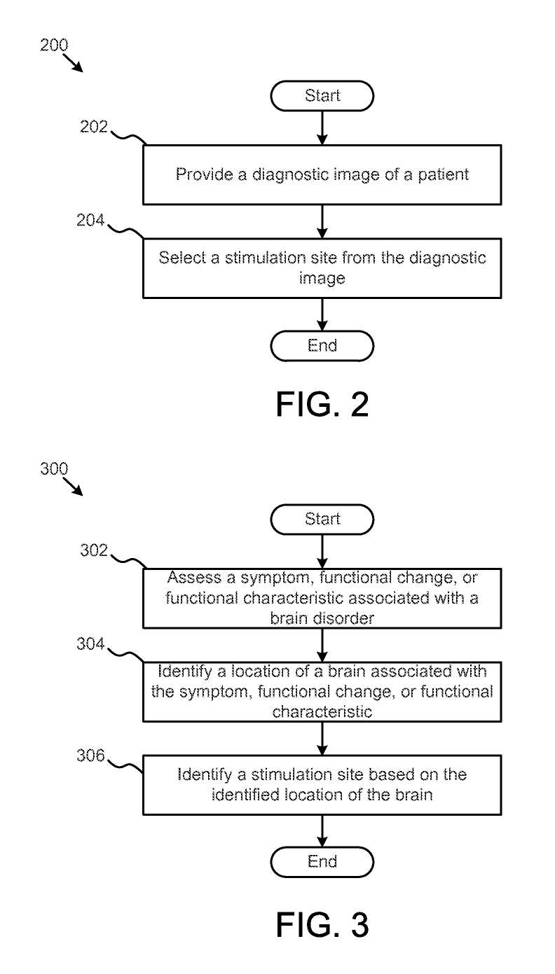

FIG. 2 shows a method 200 for identifying a stimulation site of a brain 102. In this method, a diagnostic brain image of a patient is first provided 202. The diagnostic brain image may include a map of neural activity in the brain such as a fMRI, PET, or TMS. The method 200 also includes step 204, where a stimulation site is selected from the diagnostic image 204. A stimulation site is a site where a change in the natural remapping of the brain is expected to occur. The diagnostic image may identify a portion of the brain that is damaged, or portions of the brain that may be able to learn to perform functions lost by the damaged portion.

FIG. 3 shows an alternative method, method 300, for identifying a stimulation site of a brain 102. In this method, a symptom, functional change, or functional characteristic with a brain disorder is assessed 302. For example, a particular limb may be observed to have reduced functionality due to a brain disorder. At step 304, the method includes the identification of a location of a brain associated with the symptom, functional change, or functional characteristic. For example, if a particular limb is observed to have reduced functionality, the particular portion of the brain responsible for that functionality may be identified. Finally, the stimulation site may be identified in response to identifying the location of the brain associated with the symptom, functional change, or functional characteristic.

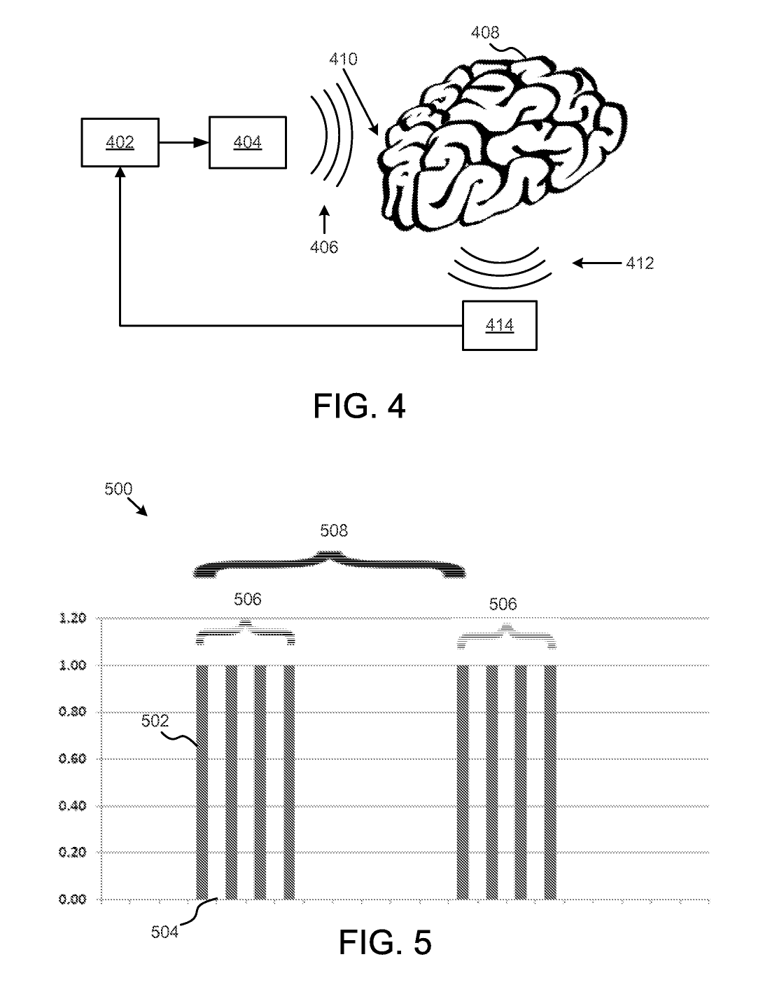

FIG. 4 shows a schematic representation of the application of ultrasound to the stimulation site. Pulse generator 402 produces electrical signals that are provided to the ultrasound transducer 404. The ultrasound transducer 404 may be a piezoelectric transducer, for example. In response to the electrical signals from the pulse generator 402, the ultrasound transducer 404 creates ultrasound signals 406 that stimulate brain 408 at the stimulation site 410. In this figure, only one ultrasound transducer is shown. However, in some embodiments, a plurality of ultrasound transducers may be used to stimulate one or more stimulation sites simultaneously. For example, two ultrasound transducers may be configured to emit ultrasound signals 406 from two different locations, and the ultrasound signals may intersect at the stimulation site 410. In some embodiments, signals 412 from the brain 408 may be detected by sensor 414, which may in turn be use by pulse generator 402 to modulate the ultrasound signals 406.

In some embodiments, the ultrasound transducer 404 may be implanted under the scalp, but outside the skull of a patient. The transducer 404 may be connected to a pulse generator 402 that is battery powered and implanted underneath the scalp. Alternatively, the pulse generator 402 may be inductively coupled to the ultrasound transducer 404 through the skin. In some embodiments, the ultrasound transducer may be implanted inside a patient's skull and provide ultrasound energy through the brain dura and/or pia. If the ultrasound transducer is implanted inside the patient's skull, there may be increased accuracy in the location and amplitude of applied ultrasound. However, implanting an ultrasound transducer increases the invasiveness of the method.

FIG. 5 shows a simplified representation 500 of ultrasound pulses that can be used to provide ultrasound energy in the methods disclosed herein. In this example, there are two sets 506 of pulses 502. The pulses 502 within a set 506 are separated by a space in time 504. In some embodiments, the pulses may be square waves as shown in FIG. 5. However the pulses may have different shapes such as a sinusoidal shape, and may vary in pulse amplitude, width, and repetition rate. The frequency of ultrasound pulses 502 may be in the range of 200 kHz to 999 kHz. Moreover, the ultrasound pulses may have a duration (on-time) in the range of 10 milliseconds to 1000 milliseconds. In some embodiments, the pulses have a duration in the range of 100 milliseconds to 300 milliseconds.

As shown in FIG. 5, the application of ultrasound pulses may not be continuous. There may be a period 508 between applications of ultrasound pulses. The period 508 may be the time between individual pulses 502, or may be the time between sets 506 of pulses 502. The period 508 may vary between about 10 milliseconds to about 10 seconds, which corresponds to a repetition rate of about 0.1 Hz to 100 Hz. In some embodiments, the repetition rate may be between 1 Hz and 30 Hz, or 5 Hz and 10 Hz. The sets 506 of pulses may have a duration between 100 and 200 milliseconds. Furthermore, the repetition rate may be between 0.01 Hz to 5 Hz.

The use of sets 506 of pulses 502 may help keep temperatures down. The frequency of pulses 502 within a set 506 may be in the range of 300 Hz and lower for neurological inhibitory effects and 3 kHz or higher for excitatory effects. An average power of less than 180 mW/cm² may achieve neurologically suppressive effects to treat maladaptive plasticity but reduce or eliminate tissue damage.

The ultrasound pulses, as described above, may raise the expected resting potential of a population of neurons at the stimulation site by approximately 10% to 80% of a difference between the expected resting potential and an action potential for the population of neurons. Therefore, neurons may become more sensitive, which improves neural plasticity.

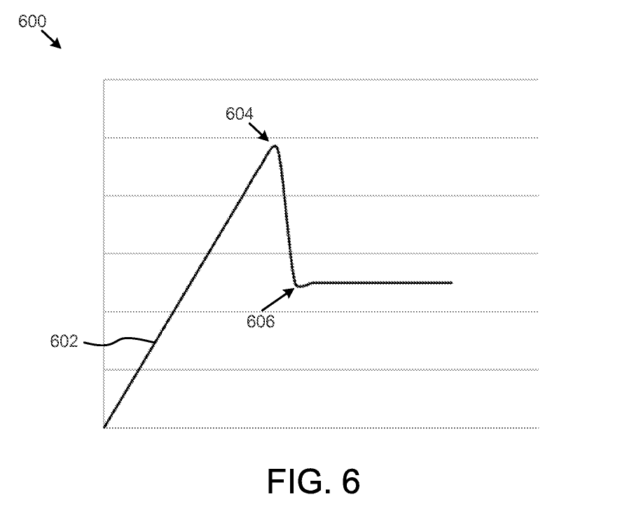

FIG. 6 shows a graph representing neurostimulation using ultrasound. The amount of ultrasound provided to a stimulation site is ramped up 602 until a the ultrasound stimulation causes a physically noticeable motor activity at the peak value 604. For example, a skeletal muscle may move in response to the ultrasound stimulation. After the motor activity is observed, the power of the ultrasound signal may be reduced. In some embodiments, the power may be reduced by about 50%, which will not produce additional motor activity, but will increase neural plasticity.

All of the methods disclosed and claimed herein can be made and executed without undue experimentation in light of the present disclosure. While the apparatus and methods of this invention have been described in terms of preferred embodiments, it will be apparent to those of skill in the art that variations may be applied to the methods and in the steps or in the sequence of steps of the method described herein without departing from the concept, spirit and scope of the invention. In addition, modifications may be made to the disclosed apparatus and components may be eliminated or substituted for the components described herein where the same or similar results would be achieved. All such similar substitutes and modifications apparent to those skilled in the art are deemed to be within the spirit, scope, and concept of the invention as defined by the appended claims.

1. A method for promoting improved brain functionality comprising:
identifying a stimulation site of a brain, where the stimulation site is associated with a brain disorder;
applying ultrasound to the stimulation site; and
initiating physical therapy.

2. The method of claim 1, where identifying the stimulation site of the brain comprises:
providing a diagnostic brain image of a patient; and
selecting a stimulation site from the diagnostic brain image.

3. The method of claim 1, where identifying the stimulation site of the brain comprises assessing a symptom, functional change, or functional characteristic associated with the brain to identify the stimulation site.

4. The method of claim 1, where applying ultrasound to the stimulation site comprises varying ultrasound parameters.

5. The method of claim 1, where applying ultrasound to the stimulation site comprises applying a plurality of ultrasound generators to a patient's head proximate to the stimulation site.

6. The method of claim 1, where applying ultrasound to the stimulation site comprises focusing ultrasound beams such that the ultrasound beams intersect at the stimulation site.

7. The method of claim 1, where applying ultrasound to the stimulation site comprises implanting ultrasound transducers under the scalp of a patient.

8. The method of claim 1, where applying ultrasound to the stimulation site comprises implanting transducers under a patient's skull bone or scalp.

9. The method of claim 1, where applying ultrasound to the stimulation site comprises providing ultrasound that is in the frequency range of 200 kHz to 10 MHz.

10. The method of claim 1, where applying ultrasound to the stimulation site comprises providing ultrasound pulses having a duration in the range of 10 milliseconds to 1000 milliseconds.

11. The method of claim 10, where the ultrasound pulses have a duration in the range of 100 milliseconds to 300 milliseconds.

12. The method of claim 1, where applying ultrasound to the stimulation site comprises repetition rates in the range of 0.1 Hz to 100 Hz.

13. The method of claim 12, where applying ultrasound to the stimulation site comprises repetition rates in the range of 1 Hz to 30 Hz.

14. The method of claim 13, where the repetition rates are in the range of 5 Hz to 10 Hz.

15. The method of claim 1, where applying ultrasound to the stimulation site comprises applying ultrasound in sets of pulses, where the pulse duration is between 100 milliseconds to 200 long and the sets are repeated at a rate of 0.01 Hz to 25 Hz.

16. The method of claim 1, where applying ultrasound to the stimulation site provides an ultrasound spatial peak temporal average (SPTA) amplitude greater than 0.01 watt/cm2 and less than or equal to 180 mW/cm2.

17. The method of claim 1, where applying ultrasound to the stimulation site raises the expected resting potential of a population of neurons at the stimulation site by approximately 10% to 80% of a difference between the expected resting potential and an action potential for the population of neurons.

18. The method of claim 1, where applying ultrasound to the stimulation site comprises:
applying ultrasound stimulation sufficient to cause a physically noticeable motor activity; and
reducing the power of the ultrasound stimulation by a predetermined amount.

19. The method of claim 1, where initiating the physical therapy occurs later in time than applying ultrasound to the stimulation site.

20. The method of claim 1, further comprising providing bioelectrodes configured to measure bioelectrical events caused by the ultrasound.
